(19) United States
(12) Patent Application Publication
(10) Pub. No.: US 2018/0117082 A1
(43) Pub. Date: May 3, 2018

(54) ANTIFUNGAL COMPOSITION/TREATMENT

(71) Applicant: THE UNIVERSITY OF NOTTINGHAM, NOTTINGHAM (GB)

(72) Inventors: SIMON AVERY, NOTTINGHAM (GB); SARA HOLLAND, NOTTINGHAM (GB); CINDY VALLIERES, NOTTINGHAM (GB); ELENA MORENO-MARTINEZ, NOTTINGHAM (GB)

(73) Assignee: THE UNIVERSITY OF NOTTINGHAM, NOTTINGHAM (GB)

(21) Appl. No.: 15/560,903

(22) PCT Filed: Mar. 18, 2016
(86) PCT No.: PCT/GB2016/050755

§ 371 Date: Sep. 22, 2017

(30) Foreign Application Priority Data
Mar. 25, 2015 (GB) 1505043.8
Dec. 21, 2015 (GB) 1522534.5

(51) Int. Cl. A61K 33/24; A01N 59/16; A61P 31/00; A61K 31/7036; A01N 43/16; A61K 31/4709; A01N 43/90; A61K 33/00; A01N 59/00; A61K 31/145; A01N 37/44; A61K 33/34; A01N 59/20; A61K 31/197; A61K 33/04; A01N 59/02

(52) U.S. Cl. CPC A61K 33/24; A01N 59/02; A61P 31/00; A61K 31/7036; A01N 43/16; A61K 31/4709; A01N 43/90; A61K 33/00; A01N 59/00; A61K 31/145; A01N 37/44; A61K 33/34; A01N 59/20; A61K 31/197; A61K 33/04; A01N 59/16

(57) ABSTRACT

An antifungal composition comprising an agent that affects the availability of functional amino acids and either i) another agent that affects the availability of functional amino acids or ii) an aminoglycoside antibiotic.

The present invention relates to antifungal compositions, and methods of treating fungal infections and/or methods of reducing fungal growth.

The need for novel antifungal treatments is significant. Pathogenic fungi occur worldwide and are major agricultural and health pests. Existing treatments are limited due to poor efficacy, expense and/or resistance among the target fungi. Fungal infections in humans are becoming a major health concern for a number of reasons, including the limited number of antifungal agents available, the increasing resistance to antifungal agents, and the growing population of immunocompromised patients at risk for opportunistic fungal infections. Crop losses in the agricultural sector due to fungal diseases pose a serious threat to the global supplies of food and fibre. Novel and effective control of fungal pathogens in the agricultural sector is therefore also needed particularly in view of such pathogens becoming resistant to commercially available antifungal treatments. There is also a need to control growth of other unwanted fungi, besides pathogens. This includes food spoilage fungi which cause significant food losses globally, and fungi that grow on commercial products and synthetic materials. Deterioration of these materials by fungi creates a significant economic burden for the industry.

An aim of the present invention is to provide an improved antifungal treatment.

According to a first aspect of the invention, there is provided an antifungal composition comprising an agent that affects the availability of functional amino acids and either i) another agent that affects the availability of functional amino acids or ii) an aminoglycoside antibiotic.

In one embodiment the antifungal composition comprises two different agents that affect the availability of functional amino acids.

In another embodiment the antifungal composition comprises an agent that affects the availability of functional amino acids and an aminoglycoside antibiotic.

Advantageously, the antifungal composition of the invention provides a synergistic inhibition of fungal growth as compared to the use of any agent in the composition alone. Preferably, the composition of the present invention is a potent antifungal treatment with a broad spectrum of activity and can be used against pathogens associated with human and agricultural fungal infections as well as other unwanted fungal growth, such as food spoilage fungi and fungi growing on other products and materials. The composition of the invention may provide up to 99% reduction in fungal growth yield at doses where either agent alone has no discernible effect on growth (for example <1% effect on growth yield). This more than 100-fold improved efficacy means that much lower amounts of either agent can be used when combined with the second agent. The combined treatment also reduces the risk of resistance development in target fungi.

Evolution of resistance to antifungals and particularly fungicides is a growing problem, underscoring the urgent need for development of new effective treatments. An advantage of combination treatments is that they reduce the likelihood of resistance: evolution of resistance to more than one agent is much slower than with a single agent. Such combinations can be particularly effective where they produce a synergistic action against the fungus. This allows lower doses of the agents to be used than if supplied singly, lessening potential concerns over non-specific toxicity or cost.

The antifungal composition of the invention may inhibit growth of unwanted fungi, for example food spoilage fungi. The antifungal composition may inhibit pathogenic fungal growth.

The term ‘antifungal’ as used herein is understood to mean the prevention and/or inhibition of the growth of fungi/fungal organisms in any environment or setting. The antifungal composition may be used amongst other things for one or more of the treatment of human or animal pathogens, or of plant pathogens or of food spoilage organisms, or of fungi that grow on synthetic materials and other commercial products.

The term “inhibit” herein is understood to mean a reduction or complete elimination of fungal growth. The reduction in fungal growth may be 100%. Alternatively, the reduction in fungal growth may be at least 90%. The reduction in fungal growth may be at least 80%. The reduction in fungal growth may be at least 70%. The reduction in fungal growth may be at least 60%. The reduction in fungal growth may be at least 50%.

The composition of the invention may decrease the minimum inhibitory concentration of the respective agents by at least 2, preferably at least 3, 4, 5, 6, 7, 8, 9, 10 or more fold. Preferably the minimum inhibitory concentration is reduced by at least 5 fold.

The antifungal composition may be a pharmaceutical composition which comprises an agent that affects the availability of functional amino acids and either i) another agent that affects the availability of functional amino acids or ii) an aminoglycoside antibiotic and a pharmaceutically acceptable excipient, diluent, adjuvant or carrier.

The pharmaceutical composition of the present invention may inhibit growth of a fungal human pathogen. The fungal human pathogen may be, among others, species of Candida (e.g. C. albicans, C. glabrata), Aspergillus, Cryptococcus (e.g. C. neoformans), Histoplasma, Pneumocystis or Stachyboirys.

The antifungal composition may be an agricultural composition which comprises an agent that affects the availability of functional amino acids and either i) another agent that affects the availability of functional amino acids or ii) an aminoglycoside antibiotic and an agriculturally acceptable support, carrier, filler and/or surfactant. An agricultural composition of the present invention may inhibit growth of a fungal plant pathogen/phytopathogenic fungi. The fungal or fungus-like plant pathogen (phytopathogenic fungi) may belong to the Ascomycete or Basidiomycete phyla, or the oomycetes. Examples include, among others, species of Botrytis (e.g. Botrytis cinerea), Pythium, Phylophthora (e.g. Phytophihora infestans), Fusarium (e,g, Fusarium graminearum), Mycosphaerella (e.g. Mycosphaerella arachidis), Rhizoctonia (e.g. Rhizoctonia solani), Thielavopsis, Sclerotinia, Cylindrocladum, Gibberella, Colletotrichium, Aspergillus (e.g. Aspergillus flavus, Aspergillus fumigatus) or Zymoseptoria (e.g. Zymoseptoria tritici).

The agricultural composition comprising the above components preferably exhibits an excellent fungicidal activity when applied to cultivated crops, for example, vegetables such as cucumbers, tomatoes, soya bean, sugar beet and eggplants; cereals such as rice, maize, wheat and barley; peas; fruit trees such as apples, pears, grapes, bananas and citrus; and potatoes, which are infected or have a possibility to be infected by harmful pathogens.

The unwanted fungus may be, but is not limited to, a food spoilage fungus. The food spoilage fungus may be, among others, species of Zygosaccharomyces (e.g. Z. rouxii, Z. bailii), Alternaria. Fusarium, Penicillium, and Cladosporium.

The unwanted fungus may be, but is not limited to, a fungus that grows on synthetic materials and other commercial products. These fungi may be, among others, Aspergillus brasiliensis, Penicillium funiculosum, Chaetomium globosum, Trichoderma virens and Aureobasidium pullulans. The composition targeting these environmental fungi preferably exhibits an excellent fungicidal activity when incorporated to polymers and other materials used in commercial products.

Where the antifungal composition is used to treat a pathogenic fungus, the fungal pathogen may be a pathogenic yeast or a pathogenic filamentous fungus. The fungal pathogen may have Sul1 and/or Sul2 proteins which have at least 40%, 50%, 60%, 70%, 80%, 90%, 95% or more sequence identity with Sul1 and/or Sul2 from Saccharomyces cerevisiae. The amino acid sequence of Sul1 from S. cerevisiae is defined as below (Seq ID no: 1):

MSRKSSTEYVHNQEDADIEVFESEYRTYRESEAAENRDGLHNGDEENWK
VNSSKQKFGVTKNELSDVLYDSIPAYEESTVTLKEYYDHSIKNNLTAKS
AGSYLVSLFPIIKWFPHYNFTWGYADLVAGITVGCVLVPQSMSYAQIAS
LSPEYGLYSSFIGAFIYSLFATSKDVCIGPVAVMSLQTAKVIAEVLKKY
PEDQTEVTAPIIATTLCLLCGIVATGLGILRLGFLVELISLNAVAGFMT
GSAFNIIWGQIPALMGYNSLVNTREATYKVVINTLKHLPNTKLDAVFGL
IPLVILYVWKWWCGTFGITLADRYYRNQPKVANRLKSFYFYAQAMRNAV
VIVVFTAISWSITRNKSSKDRPISILGTVPSGLNEVGVMKIPDGLLSNM
SSEIPASIIVLVLEHIAISKSFGRINDYKVVPDQELIAIGVTNLIGTFF
HSYPATGSFSRSALKAKCNVRTPFSGVFTGGCVLLALYCLTDAFFFIPK
ATLSAVIIHAVSDLLTSYKTTWTFWKTNPLDCISFIVTVFITVFSSIBN
GIYFAMCWSCAMLLLKQAFPAGKFLGRVEVAEVLNPTVQEDIDAVISSN
ELPNELNKQVKSTVEVLPAPEYKFSVKWVPFDHGYSRELNINTTVRPPP
PGVIVYRLGDSFTYVNCSRHYDIIFDRIKEETRRGQLITLRKKSDRPWN
DPGEWKMPDSLKSLFKFKRHSATTNSDLPISNGSSNGETYEKPLLKVVC
LDFSQVAQVDSTAVQSLVDLRKAVNRYADRQVEFHFAGIISPWIKRSLL
SVKFGTTNEEYSDDSIIAGHSSFHVAKVLKDDVDYTDEDSRISTSYSNY
ETLCAATGTNLPFFHIDIPDFSKWDV

The amino acid sequence of Sul2 from S. cerevisiae is defined as below (Seq ID no: 2):

MSREGYPNFEEVBIPDFQETNNTVPDLDDLBLEYDQYKNNENNDTFNDKD
LESNSVAKHNAVNSSKGVKGSKIDYFNPSDVSLYDNSVSQFBETTVSLKE
YYDHSIRSHLTVKGACSYLKSVFPIINWLPHYNFSWFTADLIAGITIGCV
LVPQSMSYAQVATLPAQYGLYSSFIGAYSYSFFATSKDVCIGPVAVMSLQ
TAKVIADVTAKYPDGDSAITGPVIATTLALLCGIISAAVGFLRLGFLVHL
ISLNAVAGFMTGSAFNILWGQVPALMGYNSLVNTRAATYKVVIETLKHLP
DTKLDAVFGLIPLFLLYVWKWWCGTYGPRLNDRYNSKMPRLHKIIKWTYF
YAQASRNGIIIIVFTCIGWAITRGKSKSERPISILGSVPSGLKEVGVFHV
PPGLMSKLGPNLPASIIVLLLEHIAISKSFGRINDYKVVPDQELIAIGVS
NLLGTFFNAYPATGSFSRSALKAKCNVRTPLSGLFSGSCVLLALYCLTGA
FFYIPKATLSAVIIKAVSDLIASYQTTWMFWKMNPLDFICFIVTVLITVF
ASIEDGIYFAMCWSCAMLILKVAFPAGKFLGRVEVABVTDAYVRPDSDVV
SYVSENNNGISTLBDGGEDDKESSTKYVTNSSKKIETNVQTKGFDSPSSS
ISQPRIKYHTKWIPFDHKYTRELNPDVQILPPPDGVLVYRLSESYTYLNC
SRHYNIITBEVKKVTRRGQLIRHRKKSDRPWNDPGPWEAPAFLKNLKFWK
KRENDPESMENAPSTSVDVERDDRPLLKILCLDFSQVAQTDATALQSLVD
LRKAINQYADRQVEFHFVGIISPWVKRGLISRGFGTLNEEYSDESIVAGH
TSYHVARVPQGEENPSKYSVYTASGTNLPFFHIDIPDFAKWDI

In composition of the invention comprising an aminoglycoside antibiotic, the aminoglycoside antibiotic may be selected from, but is not limited to, any of the group comprising amikacin, arbekacin, astromicin, bekanamycin, dibekacin, dihydrostreptomycin, framycetin, gentamicin, hygromycin B, isepamicin, kanamycin, neomycin, netilmicin, paromomycin, ribostamycin, sisomicin, spectinomycin, streptomycin, tobramycin or verdamicin.

In a composition of the invention comprising an agent that inhibits amino acid availability, the agent may be an inhibitor of the uptake of one or more amino acids, or an inhibitor of the uptake of substrate(s) required for amino acid synthesis (e.g. sulphate), or an inhibitor of amino acid biosynthesis, or an inhibitor of amino-acyl-tRNA synthetases, or a structural analogue of a proteinogenic amino acid which antagonises incorporation of the proteinogenic amino acids into proteins, or an agent that inactivates thiol containing amino acids, or an agent that binds amino acids.

In a composition of the invention comprising an agent that inhibits amino acid availability, the agent may be an inhibitor of the uptake of sulphate required for amino acid synthesis (also referred to as a sulphate mimetic). The inhibitor may be selected from, but not limited to, the group comprising alpha-cyanno-4-hydroxycinnamic acid, bicarbonate, chromate, 4,4′-diisothio-cyanotostilbene-2,2′-disulphonate (DIDS), 4-Acetamido-4′-isothiocyano-2,2′-disulfonic stilbene (SITS), furosemide, malonate, molybdate, nigericin, oxalate, probenicid, selenate, tetrathionate, thiosulphate, tungstate, orthovanadate and vanadate.

One of the agents that inhibits amino acid availability may be an inhibitor of the amino acid transport activity of one or more transport proteins. Examples include but are not limited to eugenol, zaragozic acid and quinine.

One of the agents that inhibits amino acid availability may be an inhibitor of a step in a biochemical pathway that yields an amino acid. Examples include but are not limited to cyprodinil, mepanipyrim or pyrimethanil, as well as iron-sulphur targeting agents like copper, paraquat, menadione, hydrogen peroxide.

One of the agents that inhibits amino acid availability may be an amino-acyl-tRNA synthetase inhibitor. Examples of amino-acyl-tRNA synthetase inhibitors include, but are not limited to, cispentacin, icofungipen, mupirocin or tavaborole.

One of the agents that inhibits amino acid availability may be a structural analogue of a proteinogenic amino acid (amino acid analogue). Examples of a structural analogue of a proteinogenic amino acid (amino acid analogue) include, but are not limited to, azetidine, canavanine, 3,4-dihydroxyphenylalanine (DOPA), ethionine, norvaline, hydroxynorvaline, fi-N-methylamino-L-alanine (BMAA), thialysine, m-tyrosine, quinine, chloroquine, amodiaquine, mefloquine, primaquine, or quinacrine.

One of the agents that inhibits amino acid availability may inactivate thiol containing amino acids. Examples of thiol targeting agents include but are not limited to carbamate- thiocarbamate- or dithiocarbamate-containing molecules such as iodopropynyl butyl carbamate (IPBC), maneb, zineb, mancozeb, thiram or ziram.

One of the agents that inhibits amino acid availability may bind to amino acids. Examples include but are not limited to copper, silver, cadmium or nickel.

The composition may comprise one or more of the following combinations of agents:
An aminoglycoside antibiotic and a sulphate mimetic. For example, one of streptomycin, paromomycin or hygromycin B, and one of bicarbonate, chromate, malonate, molybdate, oxalate, probenicid, selenate or vanadate.
An aminoglycoside antibiotic and an inhibitor of the amino acid transport activity of one or more transport proteins. For example one of streptomycin, paromomycin or hygromycin B, and one of eugenol or quinine.
An aminoglycoside antibiotic and an inhibitor of a step in a biochemical pathway that yields an amino acid. For example, one of streptomycin, paromomycin or hygromycin B combined with cyprodinil or copper.
An aminoglycoside antibiotic and a carbamate or dithiocarbamate. For example, one of streptomycin, paromomycin or hygromycin B, and one of IPBC, maneb, zineb, mancozeb, thiram or ziram.
An aminoglycoside antibiotic and an amino acid analogue. For example, one of streptomycin, paromomycin or hygromycin B, and one of DOPA, norvaline, ethionine. quinine, chloroquine, amodiaquine, mefloquine, primaquine or quinacrine.
A sulphate mimetic and an amino acid analogue. For example, one of bicarbonate, chromate, malonate, molybdate, oxalate, probenicid, selenate or vanadate, and one of DOPA, norvaline, ethionine, quinine, chloroquine, amodiaquine, mefloquine, primaquine or quinacrine.
A sulphate mimetic and an inhibitor of a step in a biochemical pathway that yields an amino acid. For example, one of bicarbonate, chromate, malonate, molybdate, oxalate, probenicid, selenate or vanadate, combined with one of cyprodinil or copper.
A sulphate mimetic and a carbamate or dithiocarbamate. For example, one of bicarbonate, chromate, malonate, molybdate, oxalate, probenicid, selenate or vanadate, and one of IPBC, maneb, zineb, mancozeb, thiram or ziram.
Two sulphate mimetics. For example, two among bicarbonate, chromate, malonate, molybdate, oxalate, probenicid, selenate and vanadate.
A carbamate or dithiocarbamate and an agent that may bind to amino acids. For example, IPBC, maneb, zineb, mancozeb, thiram or ziram, combined with copper.
A carbamate or dithiocarbamate and an amino acid analogue. For example, one of IPBC, maneb, zineb, mancozeb, thiram or ziram and one of DOPA, norvaline, ethionine, quinine, chloroquine, amodiaquine, mefloquine, primaquine or quinacrine.
An inhibitor of the amino acid transport activity of one or more transport proteins and an inhibitor of a step in a biochemical pathway that yields an amino acid. The inhibitor of the amino acid transport activity of one or more transport proteins may be eugenol or quinine and the inhibitor of the biochemical pathway may be cyprodinil or copper.
An agent that may bind to amino acids and an amino acid analogue. For example, copper and one of DOPA, norvaline ethionine, quinine, chloroquine, amodiaquine, mefloquine, primaquine or quinacrine.

A pharmaceutical composition according to the invention may be administered systemically or topically. Systemic routes of administration include oral, intravenous, intramuscular or subcutaneous injection (including into depots for long-term release), intraocular or retrobulbar, intrathecal, intraperitoneal (e.g. by intraperitoneal lavage), transpulmonary using aerosolized or nebulized drug, or transdermal. Topical routes include administration in the form of ointments, gels, salves, ophthalmic drops, ear drops, or irrigation fluids (for example, irrigation of wounds).

According to another aspect of the invention, the composition of the present invention may be used to treat or prevent fungal infections in or on the human or animal body, alternatively, compositions of the invention may be used to prevent food spoilage fungi growing on foodstuffs, or fungi growing on synthetic materials and other commercial products.

According to another aspect of the invention, there is provided a method of protecting plants from fungal infection, preferably pathogenic fungal infection, comprising applying to the plant and/or seeds thereof and/or to a substrate used for growing said plant an amount of a composition of the present invention effective to inhibit growth of or kill one or more species of fungi, preferably pathogenic fungi.

According to another aspect of the invention, there is provided a method for curatively or preventively controlling phytopathogenic fungi of crops and increasing the yield of crops characterised in that an effective and non-phytotoxic amount of a composition of the present invention is applied via seed treatment, foliar application, stem application or drench/drip application (chemigation) to the seed, the plant and/or to the fruit of the plant or to soil and/or to inert substrate, pumice, pyroclastic materials/tuff, synthetic organic substrates and/or to a liquid substrate in which the plant is growing or in which it is desired to grow.

A use concentration of the active ingredients (aminoglycoside antibiotic and/or inhibitor of amino acid availability) of the agricultural composition of the present invention may vary depending upon various conditions such as a kind of compound to be mixed, a subject crop, a use method, a formulation form, an application amount, an application time, and a kind of harmful pathogen.

The concentration used of an agent that affects the availability of functional amino acids may be from 0.2 to 1500 ppm, preferably from 0.2 to 500 ppm, and more preferably from 0.05 to 50 ppm.

In an embodiment where the composition comprises an aminoglycoside, the concentration of the aminoglycoside ingredient may be from 4 to 300 ppm, preferably from 4 to 100 ppm, and more preferably from 1 to 50 ppm.

The preferred method of applying the composition of the present invention to a plant is a foliar application (spraying, atomizing, dusting, scattering or pouring with or without a carrier). The number of applications and the rate of application depend on the risk of infestation by the fungal plant pathogen/phytopathogenic fungi.

The agricultural composition of the present invention can be further mixed with other agricultural chemicals, such as another fungicide, insecticide, miticide, nematicide, soil insect pesticide, antivirus agent, attractant, herbicide, plant growth or regulating agent.

The antifungal composition of the present invention is preferably applied wherein both agents are intimately admixed in order to ensure simultaneous administration. Administration of the agents can also be a ‘sequential-combined’ administration or application i.e. the first and second agents are administered or applied sequentially in such a way that they will necessarily become admixed together at the site to be treated. This can be achieved if sequential administration or application takes place within a short period of time, e.g. within less than 24 hours, preferably less than 12 hours.

Preferably a composition of the invention shows no synergy against plant and/or mammalian cells. Preferably a composition of the invention is not detrimental to plant and/or mammalian cells.

The skilled person will understand that optional features of one embodiment or aspect of the invention may be applicable, where appropriate, to other embodiments or aspects of the invention.

Embodiments of the invention will now be described in more detail, by way of example only, with reference to the accompanying drawings.

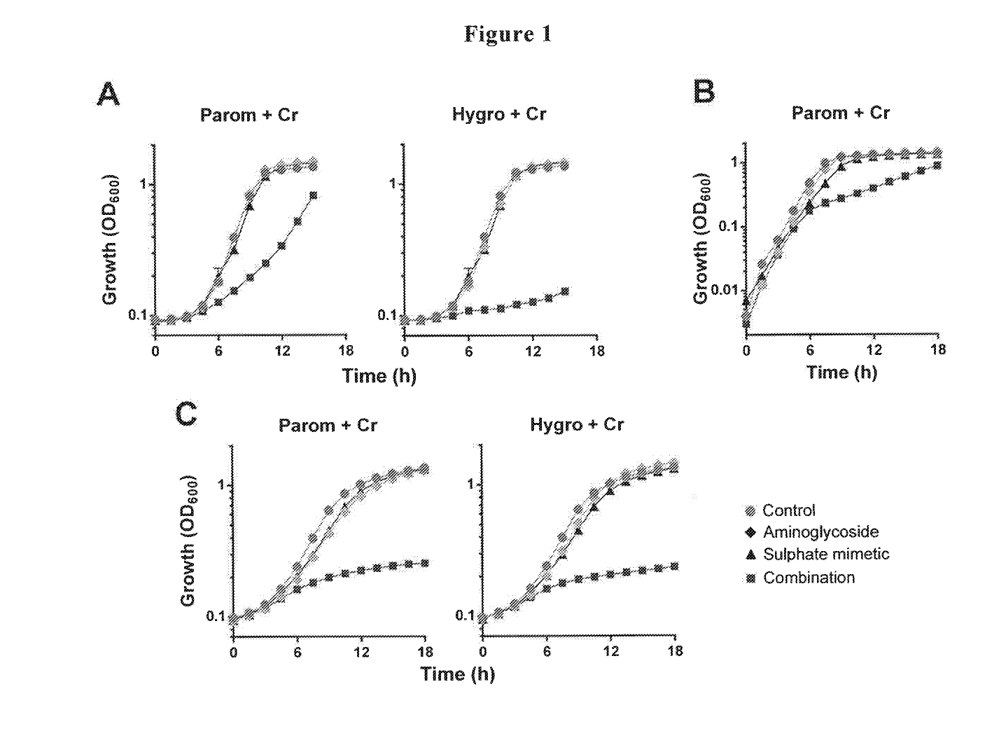

FIG. 1—illustrates the synergistic growth inhibition of fungi pathogenic to humans by aminoglycoside antibiotics and sulphate mimetics. FIG. 1A shows growth of C. albicans in YEPD broth supplemented with 200 μg ml⁻¹ paromomycin, 10 μg ml⁻¹ hygromycin B and/or 25 μM chromate. (B) Growth of C. glabrata in YEPD broth supplemented with 400 μg ml⁻¹ paromomycin and/or 50 μM chromate. (C) Growth of Cryptococcus neoformans in YEPD broth supplemented with 12.5 μg ml⁻¹ paromomycin, 0.625 μg ml hygromycin B and/or 12.5 μM chromate. Data shown are replicates from two independent cultures±SEM where these are larger than the symbol dimensions. The data for each condition are representative of at least two independent experiments performed on different days.

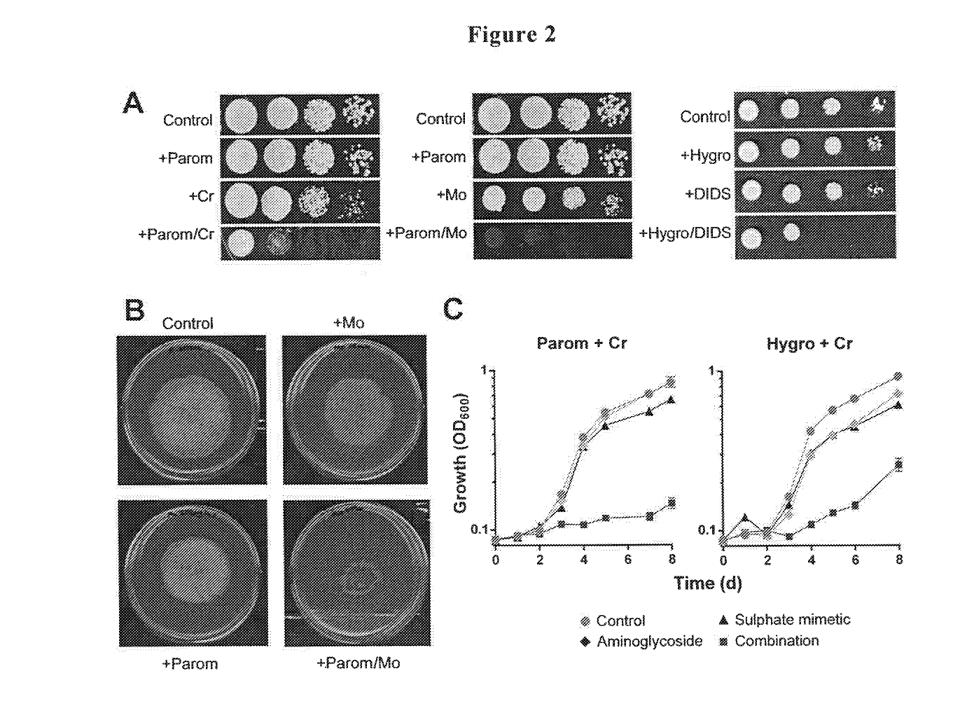

FIG. 2—illustrates the synergistic growth inhibition of other undesirable fungi by aminoglycoside antibiotics and sulphate mimetics. FIG. 1A shows growth of the food spoilage organism Z. bailii after spotting 10-fold serial dilutions of cell suspension on YEPD agar supplemented with 10 μg ml⁻¹ paromomycin, 50 μM chromate and/or 1 mM molybdate, or to YNB agar with 10 μg ml⁻¹ hygromycin B and/or 250 μM DIDS. (B) Growth of the plant pathogen R. solani on PDA agar supplemented with 300 μg ml⁻¹ paromomycin and 15 mM molybdate. (C) Growth of the plant pathogen Z. tritici in PDB medium supplemented with 0.5 μg ml⁻¹ paromomycin, 0.25 μg ml⁻¹ hygromycin B and/or 10 μM chromate. The data for each condition are representative of at least two independent experiments performed on different days.

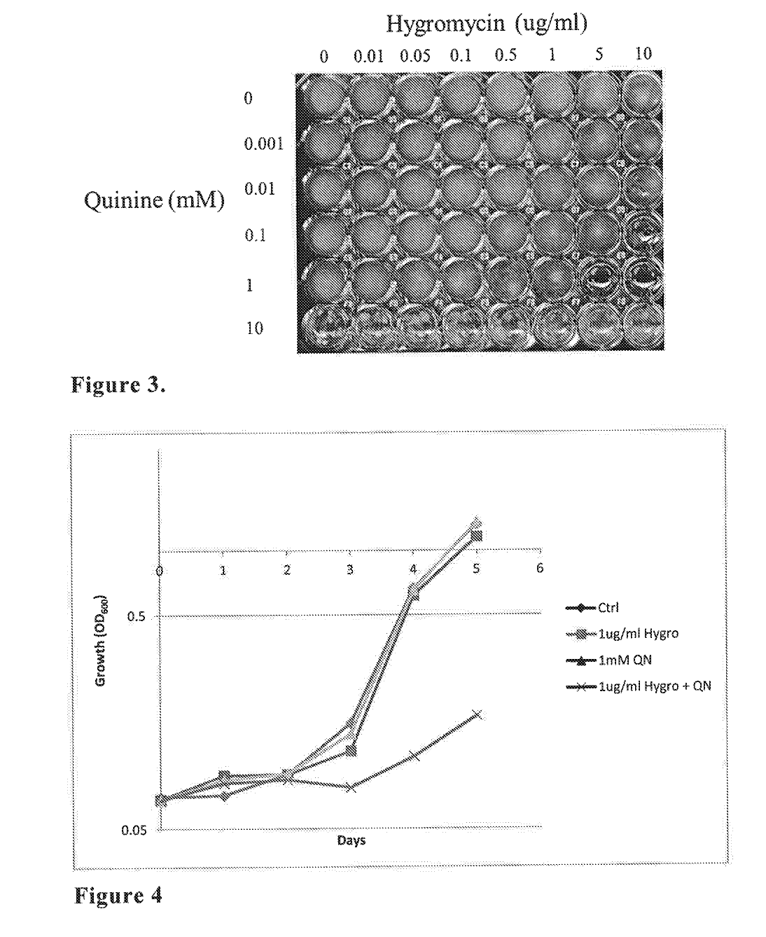

FIG. 3—illustrates the synergistic action of an aminoglycoside antibiotic and an agent that inhibits amino acid availability against the fungal plant pathogen Botrylis cinerea. The organism was cultured on Vogel's broth supplemented as indicated with hygromycin and/or quinine. The image was captured after 5 d incubation.

FIG. 4—illustrates the quantitative assessment of the synergistic action of an aminoglycoside antibiotic and an agent that inhibits amino acid availability against B. cinerea. The organism was cultured on Vogel's broth supplemented with 1 μg ml⁻¹ hygromycin and/or 1 mM quinine as indicated. Measurements of OD₆₀₀ were taken daily up to 5 d and the values shown are means from 8 replicate determinations.

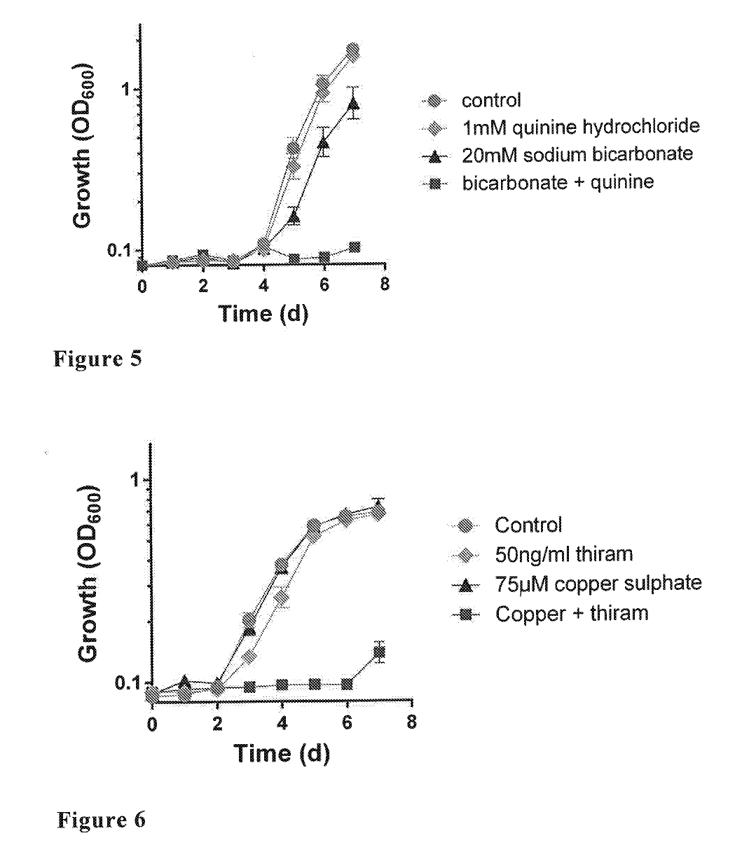

FIG. 5—illustrates the synergistic action of two different agents that inhibit amino acid availability against the fungal plant pathogen B. cinerea. The organism was cultured in Vogel's broth supplemented as indicated with bicarbonate and/or quinine. Data shown are replicates from two independent cultures±SEM where these are larger than the symbol dimensions. The data for each condition are representative of at least two independent experiments performed on different days.

FIG. 6—illustrates the synergistic action of two different agents that inhibit amino acid availability against the fungal plant pathogen R. solani. The organism was cultured in PDB medium supplemented as indicated with thiram and/or copper. Data shown are replicates from two independent cultures±SEM where these are larger than the symbol dimensions. The data for each condition are representative of at least two independent experiments performed on different days.

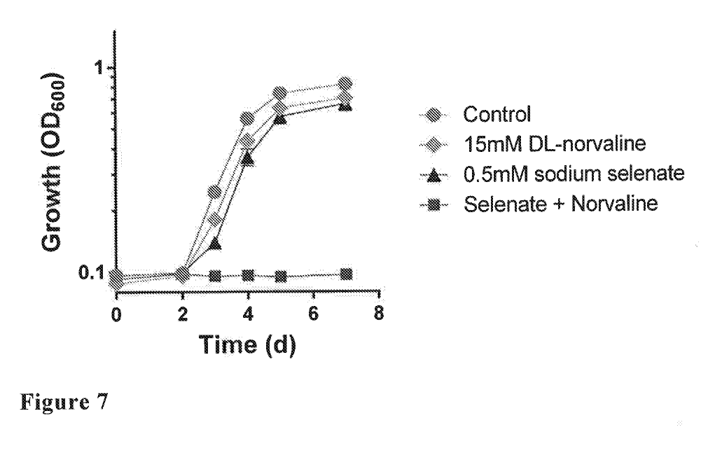

FIG. 7—illustrates the synergistic action of two more different agents that inhibit amino acid availability against the fungal plant pathogen R. solani. The organism was cultured in PDB medium supplemented as indicated with norvaline and/or selenate. Data shown are replicates from two independent cultures±SEM where these are larger than the symbol dimensions. The data for each condition are representative of at least two independent experiments performed on different days.

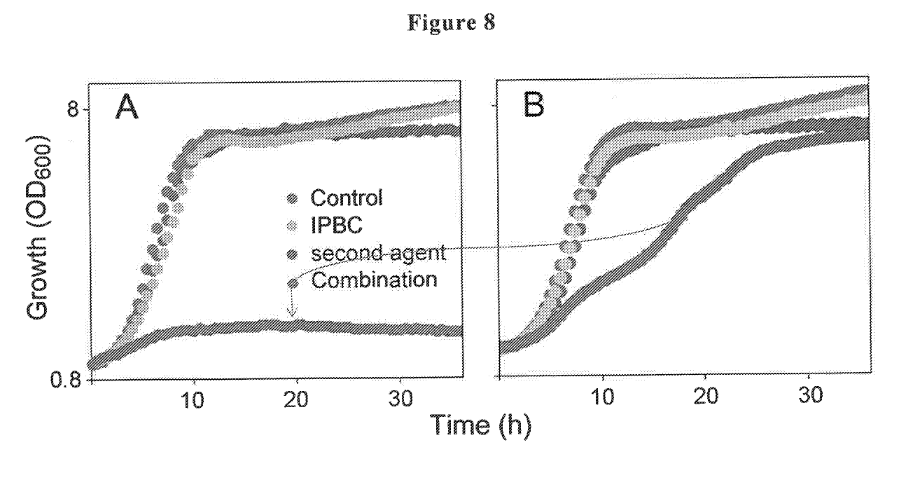

FIG. 8—illustrates synergies involving the biocide iodopropynyl-butyl-carbamate (IPBC). Saccharomyces cerevisiae was cultured with sub-inhibitory doses of IPBC (500 ng ml⁻¹) and of the second agents either 10 μg ml⁻¹ hygromycin (A) or 7 mM copper (B). Data shown are replicates from two independent cultures±SEM where these are larger than the symbol dimensions. The data for each condition are representative of at least two independent experiments performed on different days.

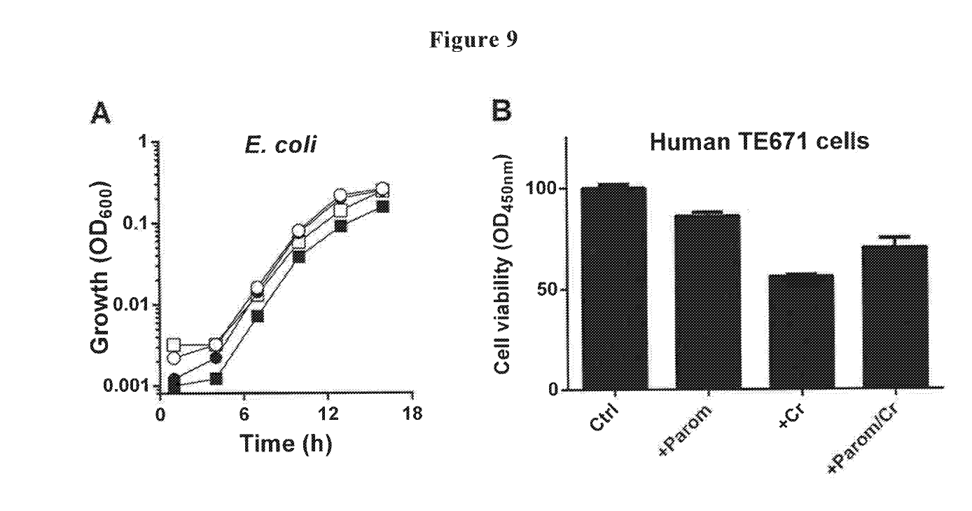

FIG. 9—illustrates the absence of synergy against non-target cells. (A) E. coli growing in LB broth supplemented without (◯) or with 1 μg ml⁻¹ paromomycin (□), 15 μM chromate (●), or both agents (▪). (B) Human cells were incubated in DMEM broth supplemented with 1 mg ml⁻¹ paromomycin and/or 10 μM chromate and relative viability was estimated from tetrazolium reduction activity. Data are representative of more than one independent experiment performed on different days.

### EXAMPLES

### Materials and Methods

### Fungal Strains and Maintenance

The pathogenic, food spoilage or laboratory fungi tested in this study were the yeasts Candida albicans, Candida glabrata, Zygosaccharomyces bailii, Saccharomyces cerevisiae and the filamentous fungi Botrytis cinerea, Rhizoctonia solani and Z. tritici, all from culture collections at the University of Nottingham. The yeasts were routinely grown and maintained on YEPD agar: 1% (w/v) yeast extract (Oxoid), 2% (w/v) peptone (Oxoid), 2% (w/v) glucose, 2% (w/v) bacteriological agar (Sigma-Aldrich). R. solani and Z. tritici were routinely maintained and grown on potato dextrose agar (PDA; Oxoid). B. cinerea was routinely maintained on malt extract agar (MEA): 2% (w/v) malt extract (Fluka Analytical), 0.6 (w/v) % peptone, 1.6% (w/v) bacteriological agar.

### Chemicals

All chemicals were from Sigma-Aldrich except hygromycin B (Panreac Applichem). Stock solutions were prepared in distilled water except for DIDS (in 0.1 M KHCO₃), quinine (in 70% ethanol) and IPBC or thiram (in DMSO). The final concentrations of KHCO₃, ethanol or DMSO in control and test media containing DIDS, quinine, IPBC or thiram were matched. All stock solutions were filter-sterilized before additions to media.

### Growth Inhibition Assays in Broth

Candida spp. or S. cerevisiae were inoculated from YEPD plates to YEPD broth (composition as above, minus agar) and cultured overnight at 30° C., 120 rev min⁻¹. Overnight cultures were diluted to OD₆₀₀˜0.5 and cultured for a further 4 h in fresh YEPD before dilution of these experimental cultures to OD₆₀₀˜0.01 or ˜0.1 in the same medium. Aliquots (300 μl) of the diluted culture plus any chemical supplements (see above), as specified, were transferred to 48-well plates (Greiner Bio-One). OD₆₀₀ was monitored continuously in a BioTek Powerwave-XS microplate spectrophotometer, with shaking at 30 C. The bacterium Escherichia coli was tested in a similar way but with growth in LB broth and at 37 C. Spores of R. solani or Z. tritici were inoculated from PDA plates to potato dextrose broth (PDB; Fluka) (20,000 spores ml⁻¹). Aliquots (150 μl) of the diluted culture plus any chemical supplements, as specified, were transferred to 96-well plates (Greiner Bio-one) and cultured over 8 d at 24° C., 120 rev min-1. OD₆₀₀ was monitored every day in a BioTek EL800 microplate spectrophotometer. Spore suspensions from Botrytis cinerea on MEA were prepared in 0.1% (v/v) tween-80. Aliquots of spore suspensions were diluted in Vogel's broth (http://www.fgse.net/methods/vogels.html) to give a final density of 10⁴ spores ml⁻¹. Aliquots (300 μl) of the diluted suspension plus any chemical supplements (see above), as specified, were incubated as above. Human TE671 cells were cultured in DMEM supplemented with 10% foetal bovine serum, L-glutamine (2 mM), penicillin (100 U ml⁻¹), streptomycin (100 μg ml⁻¹) in 25 cm² cell culture flasks, 36.5° C., 5% oxygen. Cells were detached with trypsin/EDTA and washed in 10 ml DMEM. Then 100 ul of cell suspension (in DMEM without antibiotics) were dispensed to 5000 cells/well in a 96-well plate. After 24 h, paromomycin or chromate were added as specified. After a further 24 h, 10 μl of CCK-8 reagent (Sigma) were added to each well. After 4 h incubation, formazan production was determined at 450 nm using a BioTek EL800 microplate spectrophotometer.

### Growth Inhibition Assays on Solid Medium

For qualitative growth-inhibition assays with yeasts on solid medium, experimental cultures prepared as described above were adjusted to OD₆₀₀˜2.0, 0.2, 0.02, 0.002, 0.0002 and the dilution series spotted (4 μl) on to either YEPD agar (above) or yeast nitrogen base (YNB) agar: 0.69% (w/v) yeast nitrogen base without amino acids (Formedium; Norfolk, UK) supplemented with 2% (w/v) D-glucose, 0.06 mg ml⁻¹ leucine, 0.02 mg ml⁻¹ histidine, 0.02 mg ml⁻¹ uracil and 2% (w/v) bacteriological agar (Sigma-Aldrich). Images were captured after 2 d (YEPD) or 3 d (YNB) growth at 30 C. For growth-inhibition assays on solid medium with R. solani, circular sections of ˜0.5 cm diameter were excised from cultures on PDA and transferred to the centre of fresh plates. Images were captured after 3 d growth at 28 C and the total mycelial area determined using ImageJ and GIMP2 software. Where specified, chemical supplements were included in the solid media.

### Results

The effects of different combinations of aminoglycosides with inhibitors of amino acid availability were tested in key fungi of interest. Agents were supplied at doses which, individually, were just sub-inhibitory to the fungi. Accordingly, inclusion of either 25 μM chromate, 200 μg ml⁻¹ paromomycin or 10 μg ml⁻¹ hygromycin in the growth medium had no discernible inhibitory effect on growth of the yeast Candida albicans (FIG. 1A). However, growth of this human pathogen was slowed when Cr was combined with aminoglycoside, especially hygromycin which gave ≥90% growth inhibition in combination with Cr. Checkerboard analysis (not shown) indicated that these combinations decreased the MICs of the individual agents by ≥16-fold. Synergistic growth inhibition was also evident with another pathogenic Candida species, C. glabrata, although the effect did not become apparent until mid-exponential growth (˜6 h) in this case (FIG. 1B). There was also synergistic growth inhibition of the human pathogen Cryptococcus neoformans (FIG. 1C). The food spoilage yeast Zygosaccharomyces bailii was hyper-sensitive to combinations of aminoglycoside and inhibitors of sulphate transport (which limit availability of sulphur-containing amino acids) (FIG. 2A): most dilutions of Z. bailii cell suspensions showed little growth on agar supplemented with two agents (paromomycin or hygromycin combined with Cr, Mo or DIDS), supplied at concentrations where neither agent alone had a significant effect on growth. Higher concentrations of molybdate than chromate were required to achieve this effect, reflecting the fact that molybdate is the less toxic of these two agents. Synergistic growth inhibition of the fungal plant pathogen Rhizoctonia solani was also evident (FIG. 2B). Outward growth of R. solani from a central, point inoculum on agar was compromised by incorporation of paromomycin and molybdate into the medium, supplied at concentrations where neither agent alone had a marked growth-inhibitory effect. Combinations of Cr with different aminoglycosides also produced strong, synergistic inhibition of growth of another fungal plant pathogen, Zymoseptoria tritici (FIG. 2C).

The antimalarial drug quinine has recently been shown to mimic the amino acid tryptophan and cause tryptophan starvation. In combination with hygromycin, quinine produced synergistic growth inhibition of the fungal plant pathogen Botrylis cinerea. For example, growth of B. cinerea was slowed at 5 μg ml⁻¹ hygromycin in the absence of quinine, but was strongly inhibited at 0.5 μg ml⁻¹ hygromycin in the presence of 1 mM quinine (FIG. 3). At appropriate sub-inhibitory doses of these agents, growth of B. cinerea was inhibited ≥90% when they were combined compared to growth with either agent alone (FIG. 4).

Other effective combinations did not include an aminoglycoside. Combinations of two different agents that inhibit amino acid availability also demonstrated synergistic growth inhibition. Thus, quinine combined with a sulphate transport inhibitor like bicarbonate produced synergistic growth inhibition of B. cinerea (FIG. 5). Similarly, combinations of thiram and copper (FIG. 6) or of norvaline and selenate (FIG. 7), produced striking synergistic inhibition of R. solani growth, at concentrations where none of these agents had an inhibitory effect individually. The biocide iodopropynyl-butyl-carbamate (IPBC) was tested against the yeast model S. cerevisiae, where it produced synergistic inhibition in combinations with hygromycin or copper (FIG. 8).

The invention did not affect alternative (non-fungal) cell types that were tested. Paromomycin and Cr were supplied to the bacterium E. coli at concentrations that, individually, were just sub-inhibitory to growth. The combination had no further inhibitory effect (FIG. 9A). Similarly, combination of paromomycin and Cr had no effect on mammalian cells that was not already present when these agents were supplied individually (FIG. 9B). The results indicate that compositions of the invention act on fungi with some specificity.

1. An antifungal composition comprising an agent that affects the availability of functional amino acids and either i) another agent that affects the availability of functional amino acids or ii) an aminoglycoside antibiotic.

2. An antifungal composition according to claim 1 comprising two different agents that affect the availability of functional amino acids or an agent that affects the availability of functional amino acids and an aminoglycoside antibiotic.

3. (canceled)

4. An antifungal composition according to claim 2, wherein the aminoglycoside antibiotic is selected from the group comprising amikacin, arbekacin, astromicin, bekanamycin, dibekacin, dihydrostreptomycin, framycetin, gentamicin, hygromycin B, isepamicin, kanamycin, neomycin, netilmicin, paromomycin, ribostamycin, sisomicin, spectinomycin, streptomycin, tobramycin or verdamicin.

5. An antifungal composition of claim 1, wherein the agent that inhibits amino acid availability is an inhibitor of the uptake of one or more amino acids, or an inhibitor of the uptake of substrate(s) required for amino acid synthesis, or an inhibitor of amino acid biosynthesis, or an inhibitor of amino-acyl-tRNA synthetases, or a structural analogue of a proteinogenic amino acid, or an agent that inactivates thiol containing amino acids, or an agent that binds amino acids.

6. The antifungal composition according to claim 5, wherein the inhibitor of the uptake of substrate required for amino acid synthesis is an inhibitor of the uptake of sulphate.

7. The antifungal composition according to claim 6 wherein the inhibitor of the uptake of sulphate is selected from the group comprising alpha-cyanno-4-hydroxycinnamic acid, bicarbonate, chromate, 4,4′-diisothio-cyanotostilbene-2,2′-disulphonate (DIDS), 4-Acetamido-4′-isothiocyano-2,2′-disulfonic stilbene (SITS), furosemide, malonate, molybdate, nigericin, oxalate, probenicid, selenate, tetrathionate, thiosulphate, tungstate and vanadate.

8. The antifungal composition according to claim 1 wherein the agent that inhibits amino acid availability may be an inhibitor of the amino acid transport activity of one or more transport proteins.

9. The antifungal composition according to claim 8, wherein the inhibitor of one or more transport proteins is eugenol, zaragozic acid or quinine.

10. The antifungal composition according to claim 5, wherein the inhibitor of amino acid biosynthesis is cyprodinil, mepanipyrim, pyrimethanil or copper.

11. The antifungal composition according to claim 5, wherein the inhibitor of amino-acyl-tRNA synthetases is cispentacin, icofungipen, mupirocin or tavaborole.

12. The antifungal composition according to claim 5, wherein the structural analogue of a proteinogenic amino acid is azetidine, canavanine, 3,4-dihydroxyphenylalanine (DOPA), ethionine, hydroxynorvaline, β-N-methylamino-L-alanine (BMAA), thialysine, m-tyrosine, quinine, primaquine, or chloroquine.

13. The antifungal according to claim 5, wherein the agent that inactivates thiol containing amino acids is iodopropynyl butyl carbamate (IPBC), maneb, zineb, mancozeb, thiram or ziram

14. (canceled)

15. A pharmaceutical composition or agricultural composition comprising an antifungal composition according to claim 1 and a pharmaceutically acceptable excipient, diluent, adjuvant or carrier or an agriculturally acceptable support, carrier, filler and/or surfactant, respectively.

16. (canceled)

17. Use of an antifungal composition according to claim 1, for curatively or preventatively controlling or inhibiting growth of phytopathogenic fungi.

18. Use of an antifungal composition according to claim 17, wherein the phytopathogenic fungus or fungus-like organism is of the genera Botrytis (e.g. Botrytis cinerea), Pythium, Phytophthora (e.g. Phytophthora infestans), Fusarium (e,g, Fusarium graminearum), Mycosphaerella (e.g. Mycosphaerella arachidis), Rhizoctonia (e.g. Rhizoctonia solani), Thielavopsis, Sclerotinia, Cylindrocladium, Gibberella, Colletotrichium, Aspergillus (e.g. Aspergillus flavus, Aspergillus fumigatus) or Zymoseptoria (e.g. Zymoseptoria tritici).

19. (canceled)

20. Use of an antifungal composition according to claim 1, for treating or preventing fungal infections in or on the human or animal body; or for treating or preventing fungal growth caused by a non-pathogenic fungus.

21. (canceled)

22. Use of an antifungal composition according to claim 21 wherein the non-pathogenic fungus is a food spoilage organism or is a fungus that grows on synthetic materials or other commercial product.

23. (canceled)

24. Use of an antifungal composition according to claim 20 wherein the fungal infection is caused by the species of Candida, Aspergillus, Cryptococcus, Histoplasma, Pneumocystis or Stachybotrys.

25. A method of protecting plants from fungal infection comprising applying to the plants and/or seeds thereof and/or to a substrate used for growing said plant an amount of the antifungal composition according to claim 1 to inhibit growth of or kill one or more species of fungi.

26. A method according to claim 25, wherein the species of fungus is a pathogenic fungus.

27. A method for curatively or preventively controlling phytopathogenic fungi of crops and increasing the yield of crops characterised in that an effective and non-phytotoxic amount of an antifungal composition according to claim 1 is applied via seed treatment, foliar application, stem application or drench/drip application (chemigation) to the seed, the plant and/or to the fruit of the plant or to soil and/or to inert substrate, pumice, pyroclastic materials/tuff, synthetic organic substrates and/or to a liquid substrate in which the plant is growing or in which it is desired to grow.
